(19) United States
(12) Patent Application Publication
(10) Pub. No.: US 2009/0220425 A1
(43) Pub. Date: Sep. 3, 2009

(54) Method to Quantitatively Measure Effect of Psychotropic Drugs on Sensory Discrimination

(75) Inventors: Karen Anne Moxon, Collingswood, NJ (US); Guglielmo Foffani, Philadelphia, PA (US)

Correspondence Address: Kathryn Doyle, Ph.D;Drinker, Biddle & Reath LLP, One Logan Square, 18th & Cherry Streets, Philadelphia, PA, 19103-6996, US

(73) Assignee: DREXEL UNIVERSITY, Philadelphia, PA (US)

(21) Appl. No.: 12/087,845

(22) PCT Filed: Jan. 19, 2007
(86) PCT No.: PCT/US2007/060760

§ 371 Date: Dec. 3, 2008

Related U.S. Application Data
(60) Provisional application No. 60/760,211, filed on Jan. 19, 2006.

(51) Int. Cl. A61B 5/05; A61B 10/00

(52) U.S. Cl. 424/9.2; 600/554

(57) ABSTRACT

A method for evaluating an effect of a psychotropic compound or a treatment on a neuronal activity of an animal including determining a change in the amount of information generated by neurons in response to at least one repeatedly applied stimulus, wherein the change is caused by administering the psychotropic compound or a treatment. Also provided is a method of screening psychotropic compounds for effectiveness on an animal which involves using a change in sensory discrimination in a population of neurons of the animal, wherein the sensory discrimination is obtained in response to one or more stimuli repeatedly applied to the animal and wherein a change in the sensory discrimination occurs due to administering said psychotropic compounds to the animal.

### STATEMENT REGARDING FEDERALLY SPONSORED RESEARCH OR DEVELOPMENT

This research was supported in part by U.S. Government funds (NIH grant number 2P50NS24707) and the U.S. Government may therefore have certain rights in the invention.

### FIELD OF INVENTION

The invention pertains generally to the field of psychotropic drug discovery and neuroscience. More specifically, the invention relates to methods for evaluating the effect of chemical substances or a treatment administered to an animal, particularly, psychotropic drugs on stimulus discrimination in neurons by means of information processing. Particularly, the invention relates to a method for evaluating the effect of psychotropic drugs on sensory discrimination by neurons.

### BACKGROUND OF THE INVENTION

Psychiatric disorders represent a major health problem in our society. Psychiatric patients are unable to properly process sensory information. Pharmaceutical companies are investing billions of dollars to develop and test new psychotropic drug therapies or combinations of drug therapies. However, most of these testing methods are performed in vitro and, therefore, the basic mechanisms of action of chemical substances, e.g., psychotropic drugs on stimuli processing, e.g., sensory information processing in the brain of intact animals are largely unknown, mainly due to a substantial lack of rigorous quantitative measures for evaluating the effects of these drugs on brain information processing in intact animals. This presents a problem, because the process for transferring new drugs from basic research to clinical practice is highly inefficient (i.e., too long and too expensive).

Classical neurophysiological measures of the brain's response to sensory stimulation consist of two types: the number of spikes per stimulus and the latency of the response relative to the stimulus. These simple classical measures are powerful tools to help understand which parts of the brain contribute to processing sensory information and which stimuli are the most relevant, especially when they are combined with simultaneous recording from large numbers of neurons. In general, stimuli are repeatedly presented to the animal 100, 200 and sometimes 300 times in order to evaluate the average response of a population of cells. When many different stimuli are repeatedly presented to the same cells in this way, the responses of individual cells to the broad range of stimuli can be combined to create a quantitative measure of the cells receptive field. However, recently, methods have been developed to study the response of cells to a single stimulus presentation. These data have been used to understand how ensembles of neurons encode sensory information.

Several studies in the auditory (Eggermont et al., 1981; Suga et al., 1983), visual (Movshon et al., 1978; Reid and Juraska, 1991; McLean and Waterhouse, 1994; Ringach et al., 1997) and somatosensory systems (Simons 1985; Simons and Carvell, 1989; Armstrong-James et al., 1992; Moore and Nelson, 1998; Ghazanfar and Nicolelis, 1999; Zhu and Connors, 1999; Foffani and Moxon, 2004; Tutunculer et al., 2006) have shown that cells in the infragranular cortex exhibit complex spatiotemporal firing patterns and large excitatory receptive fields. These large excitatory receptive fields may be relevant for sensory exploration of natural environments (for a review see Ghazanfar and Nicolelis, 2001).

Methods developed in the last fifteen years now allow many (>50) neurons to be simultaneously recorded, greatly increasing the power of these methods. Briefly, these methods require implanting with arrays of microwire electrodes into the infragranular layer of the somatosensory cortex. Animals are then allowed approximately one week to recover from surgery. Animals are lightly anesthetized with Nembutal. Neural signals from each wire are amplified and filtered and software is used to discriminate single neurons (e.g., Plexon, Inc, Dallas, Tex.).

Information theoretic methods have been developed to quantitatively assess the amount of information conveyed by an ensemble of neurons in response to a single stimulus presentation. The problem of understanding how ensembles of neurons code for somatosensory information has been defined as a classification problem: given the response of a population of neurons to a set of stimuli, which stimulus generated the response on a single-trial basis? Using the firing patterns of a relatively small number of sensory responsive neurons, it is possible to discriminate and classify on a single-trial basis the location of a stimulus delivered passively within the neural ensembles receptive field, for example the whisker pad in rats (Ghazanfar et al., 2000) or the paw in primates (Nicolelis et al., 1998). Importantly, it has been demonstrated that both the spatial and temporal distribution of the response properties of single neurons contribute to discrimination of sensory stimuli by ensemble of neurons. For example, even in the presence of non-dynamic stimuli, the temporal properties of the neural responses (temporal coding) provide relevant information for the discrimination that can not be found in the simple probabilities of the neurons to respond to the stimulus (rate coding) (Nicolelis et al., 1998, Ghazanfar et al., 2000, Petersen et al. 2001 a). Therefore, these information theoretic methods give an important insight into how the number of spikes per stimulus and the latency of the response to the stimulus are combined to discriminate between sensory stimuli.

The methods employed to quantitatively assess the amount of information conveyed by an ensemble of neurons in response to a single stimulus presentation vary considerably. Multivariate statistical techniques such as linear discriminant analysis (LDA) and artificial neural networks (ANNs), and different types of preprocessing stages, such as principal and independent component analysis, have been used to solve this classification problem, with surprisingly small performance differences.

U.S. Patent application US20060292549A1 to Calicos describes a device that allows the long-term stimulation of cultured neurons grown on a silicon die.

U.S. Pat. No. 6,804,661 to Cook describes a drug profiling apparatus and a method for pattern recognition and data interpretation relative to monitoring and categorizing patterns for predictably quantifying and evaluating systems of an observed entity as they react to stimuli. This patent does not describe quantifying changes in the information that an ensemble of neurons convey about a stimulus set.

Despite the current developments, there is a need in the art to develop methods of drug testing based on sensory discrimination by neurons.

All references cited herein are incorporated herein by reference in their entireties.

### BRIEF SUMMARY OF THE INVENTION

In a first aspect, the invention provides a method for evaluating an effect of a psychotropic compound on a neuronal activity of an animal, the method comprising providing the animal having at least one electrode connected with at least one area of neuronal activity, administering a psychotropic compound to the animal, repeatedly applying at least one stimulus to the animal, recording the amount of information conveyed about the at least one stimulus by (a) a population of neurons, (b) a single neuron or both, and determining a change in the amount of information generated in response to the at least one stimulus caused by said administering of the psychotropic compound.

Preferably, the stimulus is at least one of vision, auditory, touch, smell, or taste. The stimulus can be perceived by at least one of the sense of vision, auditory, touch, smell, and taste, the sense of movement, or the positional sense.

In another aspect, the invention is a method of screening psychotropic compounds for effectiveness on an animal, the method comprising using a change in sensory discrimination in a population of neurons, wherein the sensory discrimination is obtained in response to at least one stimulus repeatedly applied to the animal and wherein a change in the sensory discrimination occurs due to administering said psychotropic compounds to the animal.

In another aspect, the invention provides a method to quantitatively and rigorously measure the effects of chemicals, preferably psychotropic drugs on brain information processing. In a preferred embodiment, the information about sensory discrimination encoded by populations of neurons in the central nervous system (brain or spinal cord) is specifically modulated by psychotropic drugs. It was observed that psychotropic drugs modulate sensory discrimination at the behavioral level. This invention makes possible quantitative measures of the effects of psychotropic drugs on brain information processing in a simple animal model and provides a powerful benchmark for rigorously testing new psychotropic drugs at a very early stage of their development.

Extracting information about sensory discrimination in ensembles of neurons is preferably performed by obtaining and processing post-stimulus time histograms (PSTHs) (Foffani and Moxon, J Neurosci Methods 2004; Foffani et al., J Neurosci 2004; Tutunculer et al., Cereb Cortex 2006).

In another aspect, the invention provides a method to evaluate the effects of psychotropic drugs on sensory responses in populations of neurons by means of classical neurophysiological measures. For example, psychotropic drugs such as fluoxetine (Prozac, a selective serotonin reuptake inhibitor) and meta-chlorophenylpiperazine (MCPP, a postsynaptic serotonin receptor agonist) specifically modulate (1) the response magnitude and (2) response latency of the neurons to a sensory stimulus, thereby modulating the stimulus's neurophysiological representation.

In another aspect, the invention provides a method of evaluating the effects of psychotropic drugs on sensory discrimination in populations of neurons by means of information theory measures based on determining post-stimulus time histograms. For example, psychotropic drugs such as fluoxetine (FLU) and MCPP specifically modulate the spatial components of the neural code (i.e. information provided by spike-count) and the temporal components of the code (i.e. information provided by spike-timing), thereby increasing the informational representation of a sensory stimulus.

Inventors have discovered that using a post-stimulus time histogram (PSTH)-based method for evaluating the effects of psychotropic compound in combination with stimuli discrimination provides a new approach for an efficient drug screening procedure.

In the broadest aspect, the invention is a method for evaluating an effect of a treatment on a neuronal activity of an animal, which includes (1) providing the animal having at least one electrode connected with at least one area of neuronal activity, (2) administering a treatment to the animal, wherein the treatment is at least one of administration of a drug, administration of a substance other than a drug, electrical or magnetic stimulation such as deep brain stimulation, cortical stimulation, epidural stimulation, transcranial magnetic stimulation, transcranial direct current stimulation, electroconvulsive therapy, psychological therapy, physical therapy, surgery, or rehabilitation; (3) repeatedly applying at least one stimulus to the animal; (4) recording the amount of information conveyed about the at least one stimulus by (a) a population of neurons, (b) a single neuron, or both; and (5) determining a change in the amount of information generated in response to the stimulus caused by said administering of the treatment and thereby determining the effect of the treatment on the neuronal activity of the animal.

Inventive methods described in this disclosure can be used to correlate the change in the amount of information generated in response to the at least one stimulus caused by administering of a psychotropic compound or a treatment to a behavioral measure indicative of a sensory, motor or cognitive function in the brain.

### BRIEF DESCRIPTION OF SEVERAL VIEWS OF THE DRAWINGS

The invention will be described in conjunction with the following drawings in which like reference numerals designate like elements and wherein:

FIGS. 1A and 1B are graphs demonstrating the effects of fluoxetine (FLU), a drug known to ameliorate the effects of depression, a serotonergic re-uptake blocker (FIG. 1A) and meta-chlorophenylpiperazine (MCPP), a specific 5-HT2C receptor agonist (FIG. 1B). As can be seen from FIG. 1A, fluoxetine (FLU) dramatically increases the information encoded by the neural ensemble. Results from one ensemble of neurons recorded from a microwire array implanted in the deep layers of the forelimb rat primary somatosensory cortex in response to touch-stimuli delivered to the cutaneous surface of the contralateral body, before and after administration of FLU. FIG. 1A shows that classification performance (y-axis—bits of information) increased for every bin size tested (x-axis) after administration of FLU (black line) compared to saline (grey-line). Similar results were found with MCPP as shown in FIG. 1. Again, classification performance increase for every bin size tested after administration of MCPP (black line) compared to saline (gray-line). These results demonstrate that the effect of fluoxetine in improving the representation of sensory information by populations of neurons in the brain can be quantitatively measured. The fact that a specific 5-HT2C antagonist shows similar results, suggests that this effect could be mediated, at least in part, by activation of the 5-HT2C receptor.

FIG. 2 (prior art) is a graphic representation of a general dataset organization for a classification of S stimuli, using N neurons with B bins per neuron and T trials per stimulus as used in a PSTH-based classification method. Each row is a single-trial population response. Each column represents a variable for the classification. The PSTH of a neuron that responds to a stimulus is calculated by averaging the variables corresponding to that neuron (B columns) over the trials corresponding to the stimulus (T trials). A possible division of training-set and testing-set is represented for the first stimulus (see Foffani G, Moxon K A (2004)).

### DETAILED DESCRIPTION OF PREFERRED EMBODIMENTS OF THE INVENTION

In one aspect, the invention provides a method for evaluating an effect of a psychotropic compound on a neuronal activity of an animal. The method includes (1) providing the animal having at least one electrode connected with at least one area of neuronal activity, (2) administering a psychotropic compound to the animal, (3) repeatedly applying at least one stimulus to the animal, (4) recording the amount of information conveyed about the at least one stimulus by (a) a population of neurons, (b) a single neuron or both, and (4) determining a change in the amount of information generated in response to the at least one stimulus caused by said administering of the psychotropic compound.

In certain embodiments, recording of the amount of information is performed during at least two of time events such as (i) prior to administering the psychotropic compound to the animal, (ii) while administering the psychotropic compound to the animal and (iii) after administering the psychotropic compound to the animal.

In certain embodiments, the change in the amount of information is at least 0.1 bit. The change in the amount of information can be determined based on at least one of a response magnitude and a response latency.

In certain embodiments, the change in the amount of information is determined with the peri-stimulus time histogram (PSTH)-based classification method.

In certain embodiment, the change in the amount of information is determined with the peri-stimulus time histogram (PSTH)-based classification method or by a measure of correlation between neurons extracted with the PSTH-based classification method. In one variant of this embodiment, the change in the amount of information is determined based on single-trial analysis of the neural responses to the stimuli.

The post-stimulus time histogram (PSTH)-based method includes creating a set of templates based on the average neural responses to stimuli and classifying each single-trial by assigning it to the stimulus with the ‘closest’ template in the Euclidean distance sense. The PSTH-based method is computationally more efficient than methods as simple as linear discriminant analysis (LDA), performs significantly better than discriminant analyses (linear, quadratic or Mahalanobis) when small binsizes are used (e.g., 1 ms) and as well as LDA with any other binsize, is optimal among other minimum-distance classifiers and can be optimally applied on raw neural data without a previous stage of dimension reduction. The PSTH-based method is an efficient alternative to more sophisticated methods such as LDA and artificial neural network (ANNs) to study how ensemble of neurons code for discrete sensory stimuli, especially when datasets with many variables are used and when the time resolution of the neural code is one of the factors of interest. In a preferred embodiment, the PSTH-based method is utilized in this invention to quantitatively assess the effect of psychoactive drugs on information processing in the brain.

In another aspect, the invention is a method of screening psychotropic compounds for effectiveness on an animal, the method comprising using a change in sensory discrimination in a population of neurons, wherein the sensory discrimination is obtained in response to at least one stimulus repeatedly applied to the animal and wherein a change in the sensory discrimination occurs due to administering said psychotropic compounds to the animal.

A psychotropic compound is considered effective when due to its administering to the animal, a measurable change (e.g., 0.1 bits) in the sensory discrimination occurs. Accordingly, if an improvement in sensory discrimination has occurred, the positive value is obtained.

Preferably, the sensory discrimination is assessed by measuring the spatial and temporal components of the neural code. In certain embodiments, measuring the spatial and temporal components of the neural code is conducted by obtaining peri-stimulus time histograms (PSTHs) on populations of neurons or on a single neuron.

In a preferred embodiment, sensory discrimination is somatosensory discrimination in populations of cortical neurons or on a single cortical neuron.

In certain embodiments, the change in the sensory discrimination is determined based on at least one of a response magnitude or a response latency.

Non-limiting examples of psychotropic compounds are fluoxetine and meta-chlorophenylpiperazine.

It is to be understood that upon obtaining values for a change in sensory discrimination by the methods described in this disclosure, these values can be further analyzed to understand the effect of the psychotropic compounds on sensory, motor or cognitive function in the brain.

In the broadest aspect, the invention is a method for evaluating an effect of a treatment on a neuronal activity of an animal, which includes (1) providing the animal having at least one electrode connected with at least one area of neuronal activity, (2) administering a treatment to the animal, wherein the treatment is at least one of administration of a drug, administration of a substance other than a drug, electrical or magnetic stimulation such as deep brain stimulation, cortical stimulation, epidural stimulation, transcranial magnetic stimulation, transcranial direct current stimulation, electroconvulsive therapy, psychological therapy, physical therapy, surgery, or rehabilitation; (3) repeatedly applying at least one stimulus to the animal; (4) recording the amount of information conveyed about the at least one stimulus by (a) a population of neurons, (b) a single neuron, or both; and (5) determining a change in the amount of information generated in response to the stimulus caused by said administering of the treatment and thereby determining the effect of the treatment on the neuronal activity of the animal. The treatment is at least one of an administration of a drug, an administration of a substance other than a drug, electrical or magnetic stimulation such as deep brain stimulation, cortical stimulation, epidural stimulation, transcranial magnetic stimulation, transcranial direct current stimulation, electroconvulsive therapy, psychological therapy, physical therapy, surgery, or rehabilitation. Time events include (i) before administering the treatment to the animal, (ii) during administration of the treatment to the animal, and (iii) after administering the treatment to the animal.

This method would provide a way for drug developers or clinicians to evaluate the effect of the psychotropic compounds on the sensory, motor or cognitive functioning of the nervous system.

### DEFINITIONS

The term “single-trial analysis” as used herein denotes an analysis based on the individual responses of neurons to each individual presentation of each stimulus as opposed as the average responses of neurons to the stimuli.

The terms “psychotropic drug” or “psychotropic compound” are used herein interchangeably and denote a chemical substance that acts primarily upon the central nervous system where it alters brain function, resulting in temporary changes in perception, mood, consciousness and/or behavior.

Accordingly, as used herein, the terms “psychotropic compound or drug” include any molecule that is suspected to have psychotropic activity. Such a compound may be a small organic molecule having, for example, 50 or fewer non-hydrogen atoms, or a peptide, an oligopeptide, a nucleotide, an oligonucleotide, or a protein, typically a small protein having a molecular weight<5,000 Daltons.

Psychotropic drugs can exert effects on the central nervous system in a number of ways, including but not limited to: affecting neurons presynaptically; acting postsynaptically; and by working on neuronal axons instead of, or in addition to synapses. Mechanistically, the ways psychotropic drugs can work include: preventing the action potential from starting, e.g., by binding to voltage-gated sodium channels, so that no action potential begins even when a generator potential passes threshold; affecting neurotransmitter synthesis, e.g., by increasing synthesis of neurotransmitter precursors such as, but not limited to, L-Dopa, tryptophan, or choline, or by inhibiting synthesis of neurotransmitters such as acetyl choline (ACh); increasing or decreasing the rate of neurotransmitter packaging; increasing or decreasing release of neurotransmitters; acting as agonists by mimicking the original neurotransmitters and activating one or more associated receptors; acting as antagonists by binding to the receptor sites to block activation; preventing neurotransmitter breakdown so that it can act over a longer period of time; and by preventing reuptake. Such effects can manifest themselves in a patient as an increased sensitivity to the five senses, which may arise, for example, from an increased number of signals being sent to the brain. Assessing the influence of drugs that act in any of the foregoing ways is consistent with the practice of the present invention. Furthermore, the activity of the compounds tested relates to any of the processes of a mammalian brain, including, but not limited to: alert function, sleep, and memory formation.

Accordingly, the methods of the present invention are applicable to all drug compounds and candidate drug compounds that target brain function, including SSRIs, neuroleptics, antidepressants, antipsychotics, tranquilizers, benzodiazepines, non-phenothiazines, phenothiazines, anti-anxiety drugs, monoamine oxidase (MAO) inhibitors, sedative-hypnotics (non-barbiturate), central nervous system stimulants, anticonvulsants, non-anti-psychotic adrenergics (aromatic, non-catecholamine), as well as anxiolytics and mood stabilizers such as lithium and carbamazepine.

Non-limiting examples of psychotropic compounds include fluoxetine, meta-chlorophenylpiperazine p, methylphenidate, L-dopa, amphetamines, cathinone (khat), methylphenidate, cocaine, bupropion, diethylpropion, fluvoxamine, paroxetine, sertraline, ephedrine, pseudoephedrine, caffeine, theophylline, theobromine, clozapine, risperidone, olanzapine, quetiapine, sulpiride, ziprasidone, haloperidol, fluphenazine, thioridazine, chlorpromazine, pimozide, perphenazine, maprotiline, mirtazapine, trazodone, nicotine, betel nut, muscarine, atomoxetine, alcohol, ether, barbiturates, chloroform, chloral hydrate, methaqualone, alprazolam, diazepam, flunitrazepam, temazepam, lorazepam, opium, codeine, morphine, heroin, oxycodone, hydrocodone, methadone, fentanyl, cannabis, mescaline, psilocybin, ibogaine, nitrous oxide, ketamine, tiletamine, salvinorin, ibotenic acid, muscimol, dimenhydrinate, diphenhydramine, scopolamine, and atropine.

An area of neuronal activity in accordance with the present invention can be any ensemble of one or more neurons, and/or other excitable cells, such as muscle, heart, retinal, cochlear, tissue culture cells, stem or progenitor cells, including cell-electrode interface devices and the like. Cells can be coupled electrically, chemically, or combinations thereof. The area of neuronal activity can be an entire brain, spinal cord, ganglia, nerve, etc., or it can be a region or portion of it. Any animal source is suitable, including neural systems of invertebrates, such as mollusks, arthropods, insects, etc., vertebrates, such as mammals, humans, non-human mammals, great apes, monkeys, chimpanzees, dogs, cats, rats, mice, etc.

In preferred embodiments, the area of neuronal activity is at least one of somatosensory cortex, visual cortex, auditory cortex, olfactory cortex, premotor cortex, frontal cortex, parietal cortex, temporal cortex, thalamus, basal ganglia, striatum, hippocampus, cerebellum, spinal cord.

In certain embodiments, the area of neuronal activity include, but is not limited to, neocortex, sensory cortex, motor cortex, frontal lobe, parietal lobe, occipital lobe, temporal lobe, hypothalamus, limbic system, amygdala, septum, fornix, brain stem, medulla, pons, basal ganglia, globus pallidum, striatum, ganglion, cranial nerves, peripheral nerves, retina, cochlea, etc.

In certain embodiments, the neurons are cortical neurons in a brain of the animal. In certain embodiments, the neurons are subcortical neurons in a brain of the animal.

The term “stimulus” as used herein denotes sensory events, motor events, cognitive events, a combinations of the above, or any events which can be perceived by at least one of the sense of visual, auditory, touch, smell, and taste, the sense of movement, or the positional sense.

A stimulus can be applied to a neural system in order to elicit a response from it. The term “applied” indicates that the stimulus is administered or delivered to the system in such a way that the system reacts to it with a measurable response. The stimulus can be applied directly to the same loci where the response is measured, or it can be applied remotely at a distance from it. For instance, the stimulus can be applied on one side of a neural system (e.g., a brain), and then the response to it measured contralaterally. The stimulus set (e.g. tapping 10 locations on the paw) has to be repeatedly applied a sufficient number of times to reach a good estimate of the average responses of the neurons to the stimuli. Repetition of stimuli is necessary for performing discrimination between stimuli.

The stimulus can be of any kind, e.g., electrical, magnetic, pressure, or other force, that produces a characteristic response upon perturbation of the neural system, e.g., a brain, or structure thereof, in a pre-medicating state and a post-medicating state. It can comprise one or more components. For example, a stimulus can be an electrical stimulus presented in any effective form, e.g., as an electrical field, electrical potential difference, electric current, etc.

The term “behavioral measure” as used herein refers to a symptom, a parameter or a function indicative of a sensory, motor or cognitive function in the brain. Non-limiting examples of a behavioral measure relevant for this invention include a reaction time to a stimulus, discrimination or detection performance measured with available psychophysical tests, sensory thresholds measured with psychophysical tests, motor thresholds measured with transcranial magnetic stimulation, task performance, quantitatively measuring the ability of a subject to perform a given sensory-motor-cognitive task, quantitative measures of symptoms (e.g., using accelerometers to quantify the amplitude and frequency of tremor in Parkinson' disease), clinical scales (such as the Unified Parkinson's Disease Rating Scale (UPDRS) in Parkinson's disease), psychological tests, etc.

Any electrodes can be used for the recording. Non-limiting examples include metal, steel, activated iridium, tungsten, platinum, platinum-iridium, iridium oxide, titanium nitride, silver chloride, gold chloride based electrodes (including both microelectrodes and microwires), as well as silicon microelectronics, including tetrode or other multielectrode arrays or bundles, multichannel and ribbon devices. An exemplary electrode is described in U.S. Pat. No. 6,834,200 to Moxon et al. Activity can be measured from one or more electrodes, preferably two or more; in some cases, it may be desired to record from several regions of the neural system in order to characterize its activity.

In a preferred embodiment, the electrode is at least one of a microarray of electrodes, a micropipette, a microelectrode, a microwire, a metal electrode, a ceramic electrode, a silicon electrode, a thin-film electrode or a combination of more than one of the electrodes.

Recordings of intracellular, extracellular, or a combination thereof, can be analyzed separately, or together. The electrodes can be positioned in any arrangement which is effective to produce a suitable stimulus. Electrodes can also be external to the brain, e.g., subdural, epidural, or on the scalp. Preferably, the neural activity is recorded with at least one of single-unit recordings, multi-unit recordings, local field potential recordings, or EEG recordings.

By the term “neuronal activity,” it is meant any measurable physical behavior, output, or phenotype of the system. For example, neurons typically display variations in their membrane potential, such as action potentials, depolarizations, and hyperpolarizations. These changes in the membrane potential can be utilized as a measure of neuronal activity, e.g., by monitoring intracellularly in a single neuron, or extracellularly, the electrical activity of a single neuron or the activity of an ensemble of neurons.

The neuronal activity which is measured or assessed can be the complete neuronal activity exhibited by the system, or a subset of the total activity, e.g., a particular frequency band of the full neural signal. The measuring electrodes can detect various types of activity, e.g., spontaneous neuronal firing, slow burst activity, and background noise.

Methods for measuring and recording neuronal activity can be accomplished according to any suitable method. In certain embodiments of the invention, the neuronal activity is monitored extracellularly by measuring the extracellular electrical potential of a target population of neurons. Such measurements can reveal complex spikes or burst activity, sharp or slow waves, epileptiform spikes or seizures, arising from one or more neurons in the neural system. The neuronal activity can be measured by recording the neural system's electrical potential in the extracellular space. The electrodes used to measure the field potential produced by the neural system are referred to as “measuring electrodes” or “recording electrodes.” One or more electrodes can be used to measure the field potential. In preferred embodiments, two or more electrodes are utilized. The field potentials recorded at a given extracellular site will depend on a variety of factors, including the location of the electrode(s) with respect to the some and dendritic layers, the architecture of the neural system, the perfusion solution, etc. The signal recorded from the system can be processed to dissociate the applied field potential from the electrical activity expressed by the neurons.

The term “peri-stimulus time histogram (PSTH)-based classification” as used herein denotes an information analysis performed on the single-trial responses of neurons to individual stimuli. (see Foffani and Moxon 2004 and Foffani et al 2004). The outcome measure of the PSTH-based classification can be the percent of stimuli that are correctly classified or a more complicated information measure (e.g., bits of information, see Foffani et al., 2004). The PSTH-based classification method can also be employed to extract a measure of correlation among neurons composing the population. In another type of analysis, rather than analyzing the single-trial responses of neurons to individual stimuli, the average responses are used (multiple PSTHs) (see Tutunculer et al., Cereb Cortex 2006).

In a preferred embodiment, a PSTH-based classification method was used to quantitatively assess the information representation by ensembles of neurons in the forepaw somatosensory cortex. Ten different ensembles of neurons were recorded from five animals bilaterally implanted in the forelimb region of the primary somatosensory cortex. The average number of neurons per ensemble was 24 (range 9-39). The PSTH-based classification method was used to evaluate, for each ensemble, the ability of the neural responses to discriminate which location was stimulated on a single-trial basis. Classification performance was first expressed as the number of trials correctly classified divided by the total number of trials. For every ensemble of neurons, classification performance was greater than chance (10%) at all bin sizes. The maximal performance (52.9%, i.e., >5 times greater than chance) was obtained with an ensemble of 38 neurons at 2 msec bin size. The minimal performance (19.8%, i.e., about two times greater than chance) was obtained with an ensemble of nine neurons at 40 msec bin size. At all bin sizes there was a significant correlation (Pearson: r=0.71; p=0.02) between classification performance and the number of neurons in the ensemble. Similar results were obtained when the classification performance was expressed in bits by calculating the mutual information between predicted and actual stimuli from the confusion matrix. The two measures of classification performance (percentage of trials correctly classified and bits of mutual information) were highly correlated (Pearson: r=0.92; p=0.0001; n=70. Random neuron dropping revealed a sublinear increase of classification performance when increasing the number of neurons included in the analysis, suggesting that information about stimulus location in this simple task was redundantly distributed within the ensembles.

The ability to quantitatively measure the effects of psychoactive drugs was first tested using classical neurophysiological measures. Two rats were implanted with arrays of microwires into the infragranular layer of the primary somatosensory cortex of the rat. Animals were allowed one week to recovery. A sensory mapping procedure described herein (Tutunculer et al., 2006) was used to quantitively assess the neuronal response of the cells before and after administration of a psychotropic drug, fluoxetine (10 mg/kg). When the number of spikes per stimulus before drug was compared to the number of spikes per stimulus after drug, a significant increase in the number of spikes was observed (0.67 spikes per stimulus before drug; 1.05 spikes per stimulus after drug, p<0.001). There was also a decrease in the latency of the response, from 20 msec before drug to 17.5 msec after drug. However, this difference did not reach significance in this small sample (p=0.11). Together, these data suggest that fluoxetine increases the responseness of these cells to the stimuli (e.g., touch).

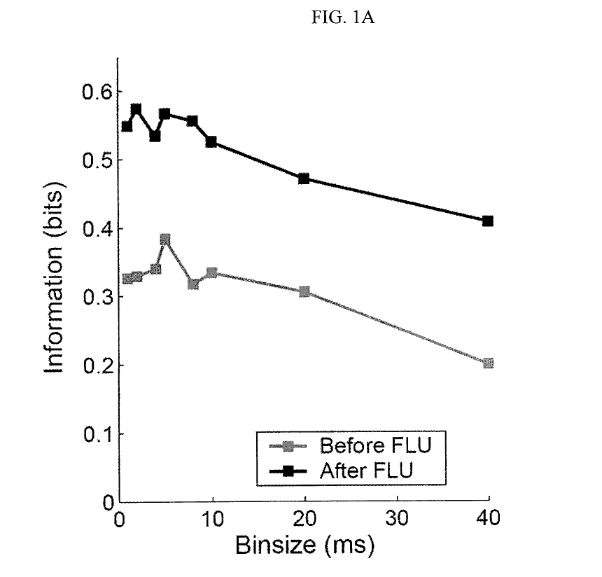

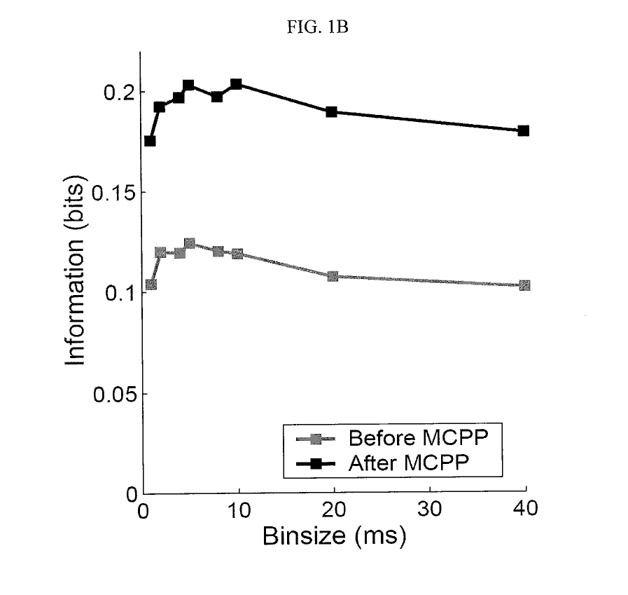

To further assess the effect of psychoactive drugs on the neuronal responses to sensory stimuli, PSTH-based classification method was used to quantitatively assess the effect of serotonergic reuptake blockers information representation (FIGS. 1A and 1B). The PSTH-based classification method was used to assess the amount of information encoded about stimulus location by the single-trial neural responses. The temporal precision of the code was investigated by evaluating how changing the binsize from 40 ms (spike-count) to 1 ms (spike-timing) (x-axis) would affect the ability of the ensemble responses to discriminate stimulus location on a single-trial basis. The data show that the amount of information represented by the ensemble was greater after administration of fluoxetine for every binsize tested.

These results demonstrate that a drug which ameliorates the symptoms of psychiatric disorders can be shown to improve the representation of sensory information by ensembles of neurons in the brain. It was also demonstrated that the amount of information per neuron was increased

Inventors have discovered that by correlating this change to a change in various symptoms during testing across a broad range of psychoactive drugs, the effectiveness of psychoactive drugs can be quantitatively measured. This invention can be used, for example, for screening drugs capable of ameliorating the sensory deficit disorder associated with many psychiatric disorders.

Inventive methods described in this disclosure can be used to correlate the change in the amount of information generated in response to the at least one stimulus caused by administering of a psychotropic compound or a treatment to a behavioral measure indicative of a sensory, motor or cognitive function in the brain.

The invention will be illustrated in more detail with reference to the following Examples, but it should be understood that the present invention is not deemed to be limited thereto.

### EXAMPLES

### Example 1

Effect of psychotropic drugs on neurophysiological measures (FIGS. 1A and 1B) Experimental design: Animals were chronically implanted with microelectrode arrays into the paw regions of the rat infragranular primary somatosensory cortex. Animals were allowed one week to recover. On the day of recording, animals were lightly anesthetized with Nembutal (0.25 mg/kg) and the neural signal from each microelectrode was filtered and amplified. Single neurons were discriminated on each channel. The responses of these single neurons were recorded while separately stimulating multiple locations on the paws, before and after administration of FLU or MCPP. The effects of these drugs on somatosensory discrimination were measured in classical neurophysiological terms by quantitatively characterizing spatial and temporal shapes of the neurons' receptive fields in terms of magnitudes and latencies of the neural responses at each location. At the conclusion of the experiment, animals are perfused through the heart and the brains are preserved for histological analysis.

Animals: Neural recordings were made in Long-Evans rats (240-300 g) with procedures approved by the Institutional Animal Care and Use Committee at Drexel University and following NIH Guidelines. Two experimental groups were used: (1) animals were recorded before and after administering FLU; (2) 10 animals were recorded before and after MCPP. These numbers were calculated to guarantee a rigorous statistical analysis according to long-term experience of the principal investigator.

Drugs: Drug administration was as follows: MCPP was obtained from Sigma (C-5554) dissolved in saline and administered intraperitoneal (IP) at doses of 0.075 (low), 0.15 (medium) and 3.0 (high) mg/kg. The low-dose has been shown to be effective. FLU was obtained from Sigma (F-132), dissolved in saline and administered by IP injection at doses of 5 (low), 10 (medium) and 20 (high) mg/kg. The medium dose was effective. Two control conditions were also used: no drug (control_1) and saline (control_2). Each of the 5 conditions (3 doses+2 controls) were tested on different days on the same animal with randomized order between animals.

Neurophysiological measures of somatosensory responses: The responses of neurons to the somatosensory stimuli were quantified by means of the peri-stimulus time histogram (PSTH). Two main measures were employed: (A) the magnitude of the responses to define the spatial shapes of the neurons' RFs and (B) the latency of the responses to define the temporal shapes of the neurons' RFs.

Statistical analyses: The significance of every neural response was assessed from the statistical distribution of the PSTH (Tutunculer et al., 2006). In order to quantitatively evaluate the neurophysiological effects of the drugs tested, magnitudes and latencies of the neural responses to the somatosensory stimuli was separately entered into a three-way analysis of variance (ANOVA). Every response of each neuron was conservatively considered as an independent sample. The first factor of the ANOVA was the body location where the stimulus was delivered, with two levels: primary receptive field (center) or secondary receptive field. The second factor was the animal group (i.e., the drug used) with two levels: FLU or MCPP. The third factor was the drug condition, with five levels: control_1 (i.e. no drug), control_2 (i.e. saline), low-dose, medium-dose, high-dose. Sheffe's test was employed for post-hoc comparisons (significance: p<0.05).

Surgical Procedures to Implant Microelectrodes. Animals were anesthetized and placed in a stereotaxic apparatus for surgery (Cartesian Research, Sandy, Oreg.). Rectangular shaped craniotomies with coordinates 0.5 mm anterior to bregma and 3.5 and 4.5 mm lateral to −1.5 posterior to bregma and 3.0 and 4.0 mm lateral (atlas of Paxinos and Watson) (Chapin and Lin, 1984) were performed bilaterally over the somatosensory forepaw (palm and digits) area to accommodate two electrode arrays (one per side), each consisting of two rows of eight 50-micron Teflon-coated stainless steel microwires (NB labs, Dennison, Tex.). The spacing between microwires before implant was approximately 200 microns. The arrays were oriented so that rows run from rostral to caudal. As each electrode array was implanted, neural activity was continuously monitored (see Single Neuron Discrimination, below for details) and amplified through auditory speakers. The forepaw was gently tapped to elicit somatosensory responses and to ensure that electrodes were properly placed in the forepaw region. When the characteristic large amplitude of layer V neurons was recorded on the majority of electrodes, the electrode was cemented in place. The connectors were surrounded with dental cement to create an electrode cap that forms a base on which to attach a recording headstage during subsequent recording sessions.

Single Neuron Discrimination. Single neuron discrimination was done 7-10 days after the implantation surgery using the same methods used to describe layer V neurons in the barrel field cortex to allow direct comparison (Ghazanfar and Nicolelis, 1999; Foffani and Moxon, 2004; Foffani et al., 2004). In brief, rats were anesthetized with low doses of Nembutal to minimize interference of the anesthesia on the neural recordings (Friedberg et al., 1999) but sufficient to immobilize the rat. Stable levels of light anesthesia were maintained at different times within the same session by giving small supplements when the rat consistently responded to tail-pinch. Signals were amplified and filtered using a multi-neuron acquisition system (Plexon inc. Dallas, Tex.) and the resulting signals were displayed on an oscilloscope and amplified through loudspeakers to aid in online neuronal spike sorting from all 32 channels (Wheeler, 1999).

Receptive Field Maps. One receptive field map was performed on each animal in the following way. Since the goal was to investigate the spatial and temporal structure of the receptive field in response to touch, 10 discrete locations were chosen for stimulation on each forelimb. These locations included one spot on each of the 5 digits, labeled (1) D1, (2) D2, (3) D3, (4) D4 and (5) D5. D3 and D4 were stimulated on the dorsal surface while digits D1, D2 and D5 were stimulated on the ventral surface. In addition, five other arbitrary but consistent locations across all animals were stimulated. These locations included a spot on (6) the dorsal paw (PAW), (7) the ventral palm (PLM), (8) the wrist (WR), (9) distal forelimb (DFL), and (10) proximal forelimb (PFL). The wrist, distal forelimb and proximal forelimb were stimulated dorsally. During stimulation, the paw and limb were placed on their side, digit 1 facing up, so that all locations were easily accessible by the stimulator. Each of the above locations was consecutively stimulated 100 times at 0.5 Hz with a fine tipped metal probe 1 mm in diameter. To be consistent with previous studies in the whisker system and ensure that only tactile receptors at the sight of contact were activated, the metal probe was controlled through a piezoelectric element actuated by a Grass stimulator (Model S48), which delivered squared-pulse stimuli (duration: 100 ms, frequency: 0.5 Hz), similar to previous studies (Chapin, 1986; Foffani et al., 2004). The tip of the metal probe moved 0.5 mm in response to the square-pulse stimuli. To control the magnitude of the stimuli at each location, the metal probe was first positioned on the skin, ensuring contact but no visual indentation under 10× magnification. The metal probe was then moved 0.5 mm away from the skin and the stimulation was started. The effect of the stimulus was viewed under 10× to ensure no movement of the digits or limb. These stimulus properties and the relatively large distance between the locations stimulated make the possibility of stimulus spread across locations extremely unlikely. All locations were stimulated within the same recording session to ensure that the same neurons were recorded in response to stimulation of all locations. All 100 stimuli were given to a location and then the stimulator was moved to the next location. There was no randomization of stimuli. The frequency of stimulation of 0.5 Hz corresponds to twice the interstimulus interval previously shown not to influence subsequent responses (Chapin, 1986). The Grass stimulator simultaneously sent pulses to the data acquisition system for precise timing of the stimulus onsets. The waveforms and action potential times of all discriminated neurons were recorded during the receptive field map and the data were stored in NeuroExplorer (Nex Technologies, Littleton, Mass., version 2.66). For every location, peri-stimulus time histograms (PSTHs, 1 ms binsize) of all the neurons were calculated using Nex functions and exported to Matlab (version 6.5, The Mathworks) for further analysis.

Quantitative Measures of Receptive Fields. In order to identify significant responses in the PSTHs, 3 tests were performed for every neuron and for every location: (1) a threshold was set as the average background activity of the neuron (evaluated from 100 ms to 5 ms before the stimulus) plus 3 standard deviations, and the first and the last significant bin (1 ms binsize) that exceeded the threshold in a window between 5 ms and 90 ms after the stimulus were identified; (2) at least 3 bins have to be over the threshold; (3) the response between the first and the last significant bin has to be significantly greater than the background activity (non-paired t-test, p<0.001). For every significant response, four parameters were extracted from the PSTH: (1) the response magnitude, defined as the integral of the PSTH between the first and the last significant bin (i.e. probability of spike per stimulus); (2) the peak response, defined as the maximum probability of spike per bin; (3) the first bin latency and (4) the peak latency, defined as the time intervals between the stimulus onset and the first significant bin or the peak, respectively. The rationale for setting the threshold as 3 standard deviations above background was to minimize the false identification of significant responses. For each neuron, the primary (or center) location of its excitatory receptive field was defined as the location that generated the greatest response magnitude. All the other locations where the neuron showed a significant excitatory response on the side of the body contralateral to the electrode were defined as secondary (or surround) locations. The locations on the ipsilateral side of the body where the neuron showed a significant excitatory response were defined as far ipsilateral (or far surround) locations. An additional parameter, the normalized response magnitude, was calculated for every neuron and for every location as the ratio between the response magnitude and the response magnitude of the primary location. Therefore, the normalized response magnitude of the primary location was equal to 1.0 by definition. The discrete receptive field size was calculated as the total number of locations where a neuron exhibited a significant response. Note that this discrete definition of receptive field size (number of locations) was dependent on the stimulation protocol and was comparable with the discrete definition typically used in whisker studies (number of whiskers) but not with the continuous definition conventionally employed in non-whisker studies (mm² of skin). To reduce errors due to the finite number of locations stimulated, neurons were included in the analyses only if the response magnitudes to stimulation of their primary location were strong enough to allow a reliable estimation of the parameters for the primary and secondary locations. Based on preliminary analyses, a threshold for the primary response magnitude was set at 0.2 spikes/stimulus. Once this minimal response magnitude for stimulation of the primary location was respected, responses to stimulation of secondary locations were considered significant according to the three criteria above, without additional thresholds (i.e. secondary response magnitudes were allowed to be less that 0.2 spikes/stimulus).

Histological Analysis. To examine the position of the electrode, post-mortem brain slices were Nissl stained to mark cell bodies. After the final recording session, rats were anesthetized and a stimulating current was placed down the electrode to mark the electrode tip (60 1A for 45 s). Each rat was transcardially perfused with 0.1 M phosphate buffer then 4% paraformaldehyde in 0.1 M phosphate buffer. The brain was removed from the skull and electrodes were extracted from the brain. The brain was stored in 40% paraformaldehyde in 0.1M phosphate buffer overnight then transferred to a 30% sucrose solution. After 5 days, the brain was removed from solution, embedded in Tek solution and frozen. The brains were cut into 40-1 m-thick coronal sections using a cryostat and collected on slides. The positions of the tips of the electrodes were easily visualized due to the hole created by the microstimulation.

### Example 2

### Effects of Psychotropic Drugs on Somatosensory Discrimination

Experimental design: The basic idea was to quantitatively measure the amount of information an ensemble of neurons can represent by using the single-trial responses of populations of neurons to identify the location on the body where each stimulus was delivered (classification performance of the ensemble). The more trials for which the stimulus location was correctly identified, the more information the ensemble can represent. To identify locations stimulated using the single-trial neural responses, the PSTH-based method was used. The classification performance of an ensemble was evaluated as bits of mutual information (Foffani et al., 2004). The effect of psychotropic drugs on two main components of classification performance was studied: (A) the spatial component provided by assessing information using a single bin encompassing the entire response (binsize=40 ms, i.e. spike-count) to construct the PSTH and (B) the temporal component provided by decreasing the bin size used to perform classification (i.e. spike-timing) (Foffani and Moxon, 2004 and Foffani et al., 2004). We expect the amount of information, or the classification performance, of the ensemble to increase after administration of drugs that activate 5-HT receptors.

Statistical analyses: The effect of drugs on the ability of ensembles to encode sensory information was assessed using a three-way ANOVA. The classification performance of each ensemble of neurons was considered as an independent sample. The first factor of the ANOVA was the spatio-temporal component of the code, with two levels: spike-count or spike-timing. The second factor was the animal group (i.e. the drug used), with two levels: FLU or MCPP. The third factor was the drug condition, with five levels: control_1 (i.e. no drug), control_2 (i.e. saline), low-dose, medium-dose, high-dose. Sheffe's test was employed for post-hoc comparisons (significance at p<0.05).

PSTH-based classification using ensembles of neurons. The responses of the ensembles of neurons to the cutaneous stimuli were used to discriminate stimulus location on a single-trial basis. The PSTH-based classification method was used. The method includes creating a set of templates based on the average neural responses to the stimuli delivered to the different locations (i.e., the PSTHs) and classifying each single-trial by assigning it to the location with the “closest” template in the Euclidean distance sense. For each animal, neurons recorded on the same side of the brain were considered as an ensemble, and single-trial responses to the 10 contralateral locations stimulated were included in the classification. All the analyses were performed in complete cross-validation by excluding from the templates only the trial to be classified and repeating the procedure for every trial. Complete cross-validation was particularly efficient using the PSTH-based classification method. For each ensemble, classification performance was evaluated by using two measures: (1) the total percentage of trials correctly classified and (2) the bits of mutual information between predicted and actual stimuli, computed from the confusion matrix of the classification (Foffani et al., 2004). Perfect classification would lead to 100% of trials correctly classified and 3.32 bits (i.e., log 2(10)) of mutual information. The upward bias of the mutual information caused by finite sampling was experimentally minimized by using 10 times as many trials per location (100) as the number of locations (10). The data processing inequality implies that the mutual information between predicted and actual stimuli was a lower bound for the mutual information between the neural responses and the actual stimuli.

Effect of Drug on the Role of spike timing. To investigate the role of spike timing and to study the temporal precision of the neural code, (1) a 40 msec poststimulus response window (5 to 44 msec) was selected, (2) this response window was divided into bins, and (3) the classification was repeated with seven different bin sizes: 1, 2, 4, 5, 10, 20, and 40 msec. A one-way repeated measure ANOVA was performed to evaluate how changing the bin size would affect classification performance. Each ensemble of neurons was considered as a different sample (n=10). The main factor of the ANOVA was the bin size, with seven levels (1, 2, 4, 5, 10, 20, and 40 msec). Tukey's honest significant difference test was used for post hoc comparisons. The ANOVA was separately performed using the two measures of classification performance introduced above.

### Example 3

### PSTH-Based Method (Foffani G, Moxon K A (2004))

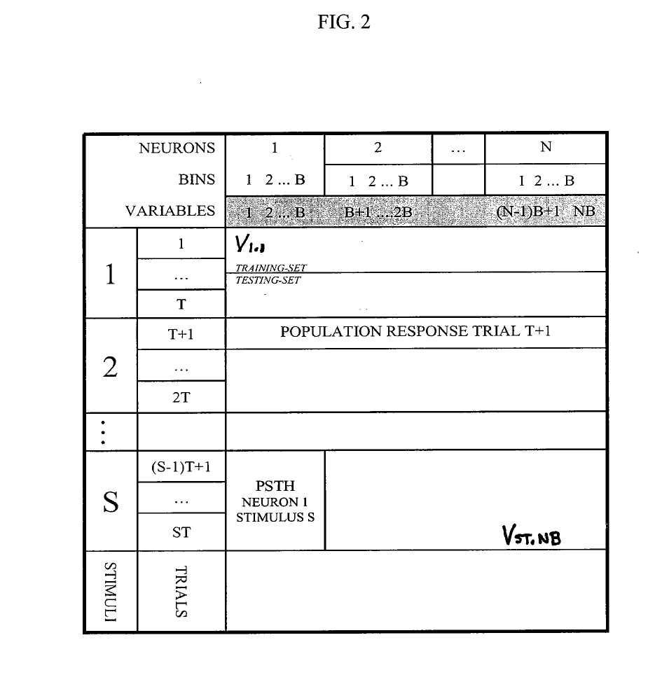

The general dataset organization is shown in FIG. 2. Single-trial neural responses are grouped in sets of S possible stimuli. Each stimulus in the set is repeated T times in the experiment, while the activity of a population of N single-neurons is recorded. For every neuron, a suitable peri-stimulus time-window that includes the response is considered. The window is divided into B bins containing spike counts with a desired temporal precision, in order to preserve timing information relative to the stimulus. In other words, the neural responses are seen as elements of a B-dimensional vector space. Therefore, the dataset is composed of a matrix with T×S rows and B×N columns. The columns are the variables and the rows are the trials, i.e. the realizations of the variables. Every element of the matrix contains the number of spikes recorded from one neuron in one trial in a specific post-stimulus bin.

Classification methods normally require two main stages: (i) a training stage, in which the variable space is divided into as many regions as the possible outputs of the classification (in our case the stimuli), and (ii) a testing stage, in which single-trials are assigned to one of the previously defined regions. As a rule, often referred as ‘cross-validation’, the training has to be performed on a subset of trials (the ‘training-set’) that is different from the subset used for the testing (the ‘testing-set’). In this disclosure, the term “complete cross-validation” is used to refer to the situation in which for every single-trial to be classified the training-set is composed by all the other trials and the testing-set is only the single-trial itself.

Data are organized in rows and columns as described above (FIG. 2). In the training stage of the PSTH-based classification method, for every stimulus a weight vector or template is defined by calculating the average number of spikes in each of the B×N variables (B=bins, N=neurons) over the training-set trials corresponding to that stimulus. Each weight vector is essentially the concatenation of the PSTHs of the neurons in the population. The weight vectors define S templates with B×N dimensions, corresponding to the S stimuli.

In the testing stage, a single-trial response of the neural population is classified as being generated by a given stimulus if the Euclidean distance between the single-trial and the template corresponding to that stimulus is minimal compared to all the other distances. The Euclidean distance is calculated by summing the square differences between each variable in the single-trial and in the template.

Three Long-Evans rats (240-300 g) were anesthetized with Nembutal (50 mg/kg i.p.) and placed in a stereotaxic apparatus for surgery (Cartesian Research, Sandy, Oreg.). The depth of anesthesia was controlled by the pinch reflex throughout the surgery and supplemental injections of 0.05 ml Nembutal were injected as necessary. The skin was incised midsagittaly. The soft tissue was retracted and the periosteum of the skull was removed. Rectangular shaped craniotomies were performed unilaterally over the whisker region of the primary somatosensory cortex from −1.0 mm to −3.0 mm posterior to bregma and 5.5 mm to 6.0 mm lateral to bregma (coordinates from atlas of Paxinos and Watson). Four burr-holes for screws and two for ground wires were drilled. Four stainless steel screws were firmly attached to the skull to ensure proper anchoring of the electrodes. Electrodes consisting of 2 rows of 8 channel 50 micron Teflon-coated stainless steel microwires (NB labs, Dennison, Tex.) were lowered slowly into the brain. Recordings were done during the implant to ensure proper placement. The electrodes were lowered to a depth of approximately 1.5 mm (layer V) and then cemented in place. The connectors were surrounded with dental cement to create an electrode cap to attach a recording headstage during subsequent recording sessions.

Passive Whisker Stimulation (Sensory Map)

After the implantation surgery, a rest period of 7-10 days was left in order to reduce the inflammatory response in the implant site. Then the rats were lightly anesthetized with Nembutal (35 mg/kg). Headstages (NB Labs, Dennison, Tex.) were attached to the electrode cap. The signals were preamplified (gain 100, bandpass 154 Hz-13 kHz) and the preamplifier outputs were connected through ribbon cables to a Multi-Neuron Acquisition Processor (MNAP) (Plexon inc. Dallas, Tex.) which enabled online neuronal spike sorting from all 32 channels implanted and simultaneous recordings during mapping. The MNAP further amplified the signals reaching a total gain of 10,000-20,000. Single neurons were discriminated on each electrode using commercial software Nex (Plexon Inc. Dallas, Tex.) (Wheeler, 1999).

Each whisker was stimulated by moving it forward approximately 5° using a fine tipped metal probe controlled by a Grass stimulator that simultaneously sent TTL pulses to the MNAP to record the precise time of stimulation. Every whisker was stimulated 100 times, with a square wave 100 ms long, at 0.5 Hz frequency. The spike times for all discriminated neurons were recorded during the experiment along with timestamps of the onset of the stimuli and a sample of the waveforms. Signals were stored in Nex (Plexon, Inc., Dallas, Tex.) and the single-trial bin counts (i.e. single-trial rate histograms, 1 ms binsize) of all the neurons were exported to Matlab (version 6.5, The Mathworks, Natick Mass., USA) for further analysis.

Data analysis I: PSTH-based classification on “raw” data.

In order to validate the PSTH-based classification method, which was coded in Matlab, the classification was first performed on raw data, without any dimension reduction. The dataset was initially organized as in FIG. 1, with S=20 (stimulated whiskers), T=100 (trials per stimulus), B=100 (bins in the post-stimulus time window) and N neurons (N=24 for rat 1, 17 for rat 2 and 15 for rat 3). Classification performance was always calculated as the percentage of trials correctly classified for every stimulus. Five levels of manipulation were applied on the data in order to test the performance of the PSTH-based classification method under different conditions, namely: ‘window selection’, ‘bin clumping’, ‘whisker dropping’, ‘trial dropping’ and ‘variable dropping’.

The first manipulation, ‘window selection’, consists of changing the size of the response window considered for the classification. The aim of this manipulation was to choose an optimal window for subsequent analyses and to test the robustness of the PSTH-based method to highly sparse datasets. The classification of 20 whiskers at 1 ms binsize was performed with two post-stimulus time windows: a 40 ms-long window (5-44 ms after contact) that contained virtually the entire on-response of the neurons (Ghazanfar et al., 2000) and a 90 ms-long window (5-94 ms after contact) that included at least 50% of the variables with little or no relevant information for the classification. A paired t-test was employed to compare the two conditions (5-44 ms vs. 5-94 ms; n=60, i.e. 20 whiskers×3 rats). Because no difference was found between the two windows (see Results), the 40 ms window was employed for all subsequent analyses, consistently with Ghazanfar et al. (2000).

The second manipulation was the ‘bin clumping’, in which the binsize was varied (1, 2, 4, 5, 8, 10, 20, 40 ms) to test the importance of temporal resolution. This manipulation is particularly important from a methodological point of view, because it affects both the neural code, in terms of temporal resolution, and the classification method, in terms of dimensionality of the dataset. The ‘bin clumping’ was performed on 3 animals in combination with ‘whisker dropping’, described in the next paragraph.

In the third manipulation, ‘whisker dropping’, the number of whiskers to be discriminated was varied in the following way: the PSTH-based classification was first applied on the complete dataset that included the 20 stimulated whiskers. Then the whiskers were sorted based on their misclassification rate, defining the ‘best’ whisker as the one with the highest number of correct classifications when stimulated and the ‘worst’ whisker as the one which when stimulated most commonly led the algorithm to select another whisker. One by one, the ‘worst’ whisker was excluded from the dataset: the PSTH template of that whisker was removed as a possible selection and the single trial tests of that whisker were eliminated. The classification was repeated until the last two whiskers remained in the dataset.

The fourth manipulation was the ‘trial dropping’, which consists of varying the number of training trials per stimulus, in order to find the optimal compromise between PSTH-based classification performance and experimental complexity. The most complete 20-whiskers classification was employed for this purpose, with 1 ms binsize. A one-way repeated measure analysis of variance (ANOVA) was executed (Statistica 5.5, Statsoft Inc., Tulsa Okla., USA) to quantitatively evaluate the performance of the PSTH-based classification with different numbers of training trials. The main factor was the number of trials, with 10 levels: T=100, 90, 80, 70, 60, 50, 40, 30, 20, 10. Classification performance was measured as the percentage of trials correctly classified (% corrects). Each whisker was treated as a different sample; therefore the sample size was n=60 (20 whiskers×3 rats). Tukey honest significant difference test was employed for post-hoc comparisons.

Finally, the ‘variable dropping’ manipulation consists of varying the number of variables (bins×neurons) used for the classification. This manipulation can be approached with the PSTH-based classification method in a very efficient way by increasing rather than dropping variables. In fact, exploiting its additive nature, the Euclidean distance dₛⁱ between the single-trial vᵢ and the templates v̅^{s} can be efficiently expressed as a function of the variables considered (se Eq. 1):

$${d}_{s}^{i} ( J ) = \sqrt{{\sum }_{j = 1}^{J} {( {v}_{i , j} - {v}_{j}^{s} )}^{2}} , with J = 1 : NB , \qquad ( Eq . 1 )$$

where s is the stimulus, i the single trial, j the index on the variables in the sum, J the number of variables employed, NB the total number of variables (N=number of neurons, B=number of bins per neuron). The classification performance is then evaluated as a function of J and the analysis only needs to be performed once. Note that evaluating dₛⁱ(J) at multiples of B corresponds to adding (or dropping) neurons in the classification. This ‘cumulative classification’ was performed on the discrimination of 20 whiskers with 1 ms binsize, using the 3 population of 24, 17 and 15 neurons. In order to be able to average the results from the 3 populations, the maximum number of neuron used in the analysis was 15.

While the invention has been described in detail and with reference to specific examples thereof, it will be apparent to one skilled in the art that various changes and modifications can be made therein without departing from the spirit and scope thereof.

### REFERENCES

Armstrong-James M, Fox K, Das-Gupta A (1992) Flow of excitation within rat barrel cortex on striking a single vibrissa. J Neurophysiol 68:1345-1358.
Bar-Gad I, Ritov Y, Bergman H (2001) The neuronal refractory period causes a short-term peak in the autocorrelation function. J Neurosci Methods 104: 155-163.
Eggermont J J, Aertsen A M, Hermes D J, Johannesma P I (1981) Spectro-temporal characterization of auditory neurons: redundant or necessary. Hear Res 5:109-121.
Foffani G, Moxon K A (2004) PSTH-based classification of sensory stimuli using ensembles of single neurons. J Neurosci Methods 135:107-120.
Foffani G, Tutunculer B, Moxon K A (2004) Role of spike timing in the forelimb somatosensory cortex of the rat. J Neurosci 24:7266-7271.
Ghazanfar A A, Nicolelis M A (1999) Spatiotemporal properties of layer V neurons of the rat primary somatosensory cortex. Cereb Cortex 9:348-361.
Ghazanfar A A, Nicolelis M A (2001) Feature article: the structure and function of dynamic cortical and thalamic receptive fields. Cereb Cortex 11:183-193.
Ghazanfar A A, Stambaugh C R, Nicolelis M A. Encoding of tactile stimulus location by somatosensory thalamocortical ensembles. J. Neurosci., 2000; 20: 3761-3775.
McLean J, Waterhouse B D (1994) Noradrenergic modulation of cat area 17 neuronal responses to moving visual stimuli. Brain Res 667:83-97.
Moore C I, Nelson S B (1998) Spatio-temporal subthreshold receptive fields in the vibrissa representation of rat primary somatosensory cortex. J Neurophysiol 80:2882-2892.
Movshon J A, Thompson I D, Tolhurst D J (1978) Spatial and temporal contrast sensitivity of neurones in areas 17 and 18 of the cat's visual cortex. J Physiol 283:101-120.
Nicolelis M A, Ghazanfar A A, Stambaugh C R, Oliveira L M, Laubach M, Chapin J K, Nelson R J, Kaas J H. Simultaneous encoding of tactile information by three primate cortical areas. Nat. Neurosci., 1998; 1: 621-630.
Petersen R S, Panzeri S, Diamond M E (2001) Population coding of stimulus location in rat somatosensory cortex. Neuron 32: 503-514.
Reid S N, Juraska J M (1991) The cytoarchitectonic boundaries of the monocular and binocular areas of the rat primary visual cortex. Brain Res 563:293-296.
Ringach D L, Hawken M J, Shapley R (1997) Dynamics of orientation tuning in macaque primary visual cortex. Nature 387:281-284.
Simons D J (1985) Temporal and spatial integration in the rat SI vibrissa cortex. J Neurophysiol 54:615-635.
Simons D J, Carvell G E (1989) Thalamocortical response transformation in the rat vibrissa/barrel system. J Neurophysiol 61:311-330.
Suga N, O'Neill W E, Kujirai K, Manabe T (1983) Specificity of combination-sensitive neurons for processing of complex biosonar signals in auditory cortex of the mustached bat. J Neurophysiol 49:1573-1626.
Tutunculer B, Foffani G, Himes B T, Moxon K A. Structure of the Excitatory RFs of Infragranular Forelimb Neurons in the Rat Primary Somatosensory Cortex Responding To Touch. Cereb Cortex 16:791-810 (2006).
Zhu J J, Connors B W (1999) Intrinsic firing patterns and whisker-evoked synaptic responses of neurons in the rat barrel cortex. J Neurophysiol 81:1171-1183.

What is claimed is:

1. A method for evaluating an effect of a psychotropic compound on a neuronal activity of an animal, the method comprising:
providing the animal having at least one electrode connected with at least one area of neuronal activity;
administering a psychotropic compound to the animal;
repeatedly applying at least one stimulus to the animal;
recording the amount of information conveyed about the at least one stimulus by (a) a population of neurons, (b) a single neuron or both; and
determining a change in the amount of information generated in response to the at least one stimulus caused by said administering of the psychotropic compound and thereby determining the effect of the compound on the neuronal activity of the animal.

2. The method of claim 1, wherein said recording of the amount of information is performed during at least two of time events, wherein said time events are selected from the groups consisting of (i) prior to administering the psychotropic compound to the animal, (ii) while administering the psychotropic compound to the animal and (iii) after administering the psychotropic compound to the animal.

3. The method of claim 1, wherein the area of neuronal activity is at least one of somatosensory cortex, visual cortex, auditory cortex, olfactory cortex, premotor cortex, frontal cortex, parietal cortex, temporal cortex, thalamus, basal ganglia, striatum, hippocampus, cerebellum, or spinal cord.

4. The method of claim 1, wherein the neurons are cortical neurons in a brain of the animal.

5. The method of claim 1, wherein the neurons are subcortical neurons in a brain of the animal.

6. The method of claim 1, wherein the stimulus is at least one of vision, auditory, touch, smell, or taste.

7. The method of claim 1, wherein the stimulus can be perceived by at least one of the sense of vision, auditory, touch, smell, and taste, the sense of movement, or the positional sense.

8. The method of claim 1, wherein the change in the amount of information is at least 0.1 bits.

9. The method of claim 1, wherein the change in the amount of information is determined based on at least one of a response magnitude and a response latency.

10. The method of claim 1, wherein the electrode is at least one of a microarray of electrodes, a micropipette, a microelectrode, a microwire, a metal electrode, a ceramic electrode, a silicon electrode, a thin-film electrode or a combination of more than one of the electrodes.

11. The method of claim 1, wherein the change in the amount of information is determined based on single-trial analysis of the neural responses to the stimuli.

12. The method of claim 1, wherein the change in the amount of information is determined with the peri-stimulus time histogram based classification method.

13. The method of claim 1, wherein the change in the amount of information is determined by a measure of correlation between neurons extracted with the peri-stimulus time histogram based classification method.

14. The method of claim 12, wherein the change in the amount of information is determined by a single-trial analysis of the neural responses to the one or more stimuli.

15. The method of claim 1, wherein the one or more stimuli are at least one of a sensory event, a motor event, a cognitive event or a combination thereof.

16. The method of claim 1, wherein the neural activity is recorded with at least one of single-unit recordings, multi-unit recordings, local field potential recordings, or electroencephalogram recordings.

17. The method of claim 1, wherein the neural activity is recorded in humans.

18. A method of screening psychotropic compounds for effectiveness on an animal, the method comprising using a change in sensory discrimination in a population of neurons, wherein the sensory discrimination is obtained in response to at least one stimulus repeatedly applied to the animal and wherein a change in the sensory discrimination occurs due to administering said psychotropic compounds to the animal.

19. The method of claim 18, wherein said sensory discrimination is assessed by measuring the spatial and temporal components of the neural code.

20. The method of claim 19, wherein said measuring the spatial and temporal components of the neural code is conducted by obtaining peri-stimulus time histograms on populations of neurons or on a single neuron.

21. The method of claim 18, wherein said sensory discrimination is somatosensory discrimination in populations of cortical neurons or on a single cortical neuron.

22. The method of claim 18, wherein the change in the sensory discrimination is determined based on at least one of a response magnitude or a response latency.

23. A method for evaluating an effect of a treatment on a neuronal activity of an animal, the method comprising:
providing the animal having at least one electrode connected with at least one area of neuronal activity;
administering a treatment to the animal, wherein the treatment is at least one of administration of a drug, administration of a substance other than a drug, electrical or magnetic stimulation such as deep brain stimulation, cortical stimulation, epidural stimulation, transcranial magnetic stimulation, transcranial direct current stimulation, electroconvulsive therapy, psychological therapy, physical therapy, surgery, or rehabilitation;
repeatedly applying at least one stimulus to the animal;
recording the amount of information conveyed about the at least one stimulus by (a) a population of neurons, (b) a single neuron, or both; and
determining a change in the amount of information generated in response to the stimulus caused by said administering of the treatment and thereby determining the effect of the treatment on the neuronal activity of the animal.

24. The method of claim 23, wherein said recording of the amount of information is performed during at least two of time events, wherein said time events are selected from the groups consisting of (i) before administering the treatment to the animal, (ii) during administration of the treatment to the animal, and (iii) after administering the treatment to the animal

25. The method claim 23, wherein the neurons are cortical neurons in a brain of the animal.

26. The method of claim 23, wherein the neurons are subcortical neurons in a brain of the animal.

27. The method of claim 23, wherein the stimulus is at least one of visual, auditory, touch, smell, or taste.

28. The method of claim 23, wherein the stimulus can be perceived by at least one of the sense of vision, auditory, touch, smell, and taste, the sense of movement, or the positional sense.

29. The method of claim 23, wherein the change in the amount of information is at least 0.1 bits.

30. The method of claim 23, wherein the change in the amount of information is determined based on at least one of a response magnitude and a response latency.

31. The method of claim 23, wherein the electrode is at least one of a microarray of electrodes, a micropipette, a microelectrode, a microwire, a metal electrode, a ceramic electrode, a silicon electrode, a thin-film electrode or a combination of more than one electrode.

32. The method of claim 23, wherein the change in the amount of information is determined on single-trial analysis of the neural responses to the stimulus.

33. The method of claim 23, wherein the change in the amount of information is determined with the peri-stimulus time histogram based classification method.

34. The method of claim 23, wherein the change in the amount of information is determined by a measure of correlation between neurons extracted with the peri-stimulus time histogram based classification method.

35. The method of claim 34, wherein the change in the amount of information is determined by single-trial analysis of the neural responses to the one or more stimuli.

36. The method of claim 23, wherein the at least one stimulus is at least one of a sensory event, a motor event, a cognitive event or a combination thereof.

37. The method of claim 23, wherein the neural activity is recorded with at least one of single-unit recordings, multi-unit recordings, local field potential recordings, or electroencephalogram recordings.

38. The method of claim 12, wherein said determining a change in the amount of information generated in response to the stimuli comprises determining a change in at least one of (i) a percent of stimuli that are correctly classified or (ii) bits of information.

39. The method of claim 38, wherein the change in the percent of stimuli that are correctly classified is at least 0.1%.

40. The method of claim 38, wherein the change in the bits of information is at least 0.1 bits.

41. The method of claim 1, further comprising correlating the change to a behavioral measure indicative of a sensory, motor or cognitive function in the brain.

42. The method of claim 23, further comprising correlating the change to a behavioral measure indicative of a sensory, motor or cognitive function in the brain.
